(19) United States
(12) Plant Patent Application Publication
(10) Pub. No.: US 2016/0174428 P1
(43) Pub. Date: Jun. 16, 2016

(54) Apple tree named 'KIZURI'

(50) ;

(71) Applicant: BETTER3FRUIT, N.V., Heverlee (BE)

(72) Inventors: Inge De Wit, Lovenjoel (BE); Hendrik Eyssen, Holsbeek (BE); Johan Keulemans, Korbeek-Lo (BE); Johan Nicolaï, Sint-Truiden (BE); Paul Van Laer, Heks (BE)

(73) Assignee: BETTER3FRUIT, N.V., Heverlee (BE)

(21) Appl. No.: 14/757,271

(22) Filed: Dec. 11, 2015

(30) Foreign Application Priority Data
Dec. 15, 2014 (QZ) 2014/3424

(51) Int. Cl. A01H 5/00

(52) U.S. Cl. PLT/162

(57) ABSTRACT

A new and distinct variety of Malus domestica apple tree named ‘KIZURI’, particularly characterized by very aromatic and sweet fruit with a solid, vibrant red flush, very firm with dense texture and very good storability.

### LATIN NAME OF THE GENUS AND SPECIES OF THE PLANT CLAIMED

Malus domestica Borkh.

### VARIETY DENOMINATION

‘KIZURI’

### PRIORITY CLAIM

This application claims priority under 35 U.S.C. § 119(f) to European Community Plant Variety Office Application No. 2014/3424 filed Dec. 15, 2014.

### BACKGROUND OF THE INVENTION

The present invention relates to a new and distinct variety of Apple tree, botanically known as Malus domestica Borkh. of the Rosaceae family, and hereinafter referred to by the variety denomination ‘KIZURI’.

The new Malus variety is a product of a controlled breeding program conducted by the inventors, Inge De Wit, Hendrik Eyssen, Johan Keulemans, Johan NicolaI and Paul Van Laer in Belgium. The objective of the breeding program was to develop a new Malus variety with crisp and juicy flesh, as well as good storage capacity and shelf life.

The new Malus variety originated from a cross made by the inventors in 1990 in Belgium. The female or seed parent is the Malus domestica variety designated ‘GOLDEN DELICIOUS’ (unpatented). The male or pollen parent is the Malus domestica variety designated ‘NY75413-30’ (unpatented). The new Malus variety was discovered and selected by the inventors within the progeny of the stated cross in a controlled environment in 2001 in Belgium.

Asexual reproduction of the new Malus variety by grafting onto rootstocks was first performed in the winter of 2002-2003 in Belgium, and has demonstrated that the combination of characteristics as herein disclosed for the new variety are firmly fixed and retained through successive generations of asexual reproduction. The new variety reproduces true to type.

### BRIEF DESCRIPTION OF THE INVENTION

The following traits have been repeatedly observed and are determined to be unique characteristics of ‘KIZURI’ which in combination distinguish this Apple tree as a new and distinct variety:

1. very aromatic fruit;

2. sweet fruit;

3. fruit with a solid, vibrant red flush;

4. very firm fruit with dense texture; and

5. very good storability.

In comparison to the parental varieties, ‘GOLDEN DELICIOUS’ (unpatented) and ‘NY75413-30’ (unpatented), ‘KIZURI’ differs primarily in the traits listed in Table 1.

TABLE 1

| | | Female Parent | |
|---|---|---|---|
| | | ‘GOLDEN | Male Parent |
| | New Variety | DELICIOUS’ | ‘NY75413-30’ |
| Trait | ‘KIZURI’ | (unpatented) | (unpatented) |
| Fruit: hue of over | red | none | dark red |
| color | | | |
| Fruit: shape | globose | oblong | oblate |
| Fruit: taste | very sweet | slightly sweet | astringent |
| Fruit: aroma | strong | low | |
| Fruit: time for | 10 days after | — | same as Golden |
| harvest | Golden | | |
| Fruit: firmness | high | medium | |
| Fruit: size | large | medium | very large |

Of the many commercial varieties known to the present inventor(s), the most similar in comparison to the new Malus variety ‘KIZURI’ is the Malus variety ‘Liberty’ (unpatented), in the following characteristics described in Table 2:

TABLE 2

| | | Comparison Variety |
|---|---|---|
| | New Variety | ‘LIBERTY’ |
| Characteristic | ‘KIZURI’ | (unpatented) |
| Fruit: ribbing | moderate | strong |
| Fruit: bloom of skin | moderate | absent |
| Fruit: pattern of over color | only solid flush | solid flush with weakly |
| | | defined stripes |
| Fruit: lenticels | present, but not very | eye-catching |
| | noticeable | |

### BRIEF DESCRIPTION OF THE PHOTOGRAPHS

The accompanying photographs illustrate the overall appearance of the new Malus variety ‘KIZURI’ showing the colors as true as is reasonably possible with colored reproductions of this type. Colors in the photographs may differ slightly from the color values cited in the detailed botanical description, which accurately describe the color of ‘KIZURI’.

FIG. 1—shows a close-up view (side, top and bottom) of mature fruit of ‘KIZURI’.

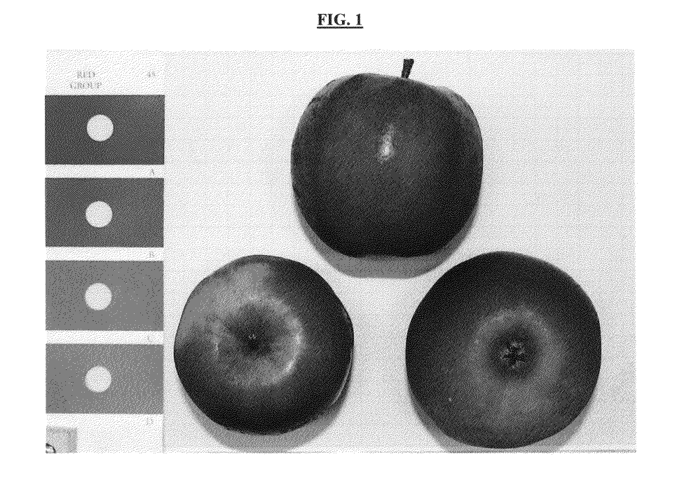

FIG. 2—shows dissection views of mature fruit of ‘KIZURI’.

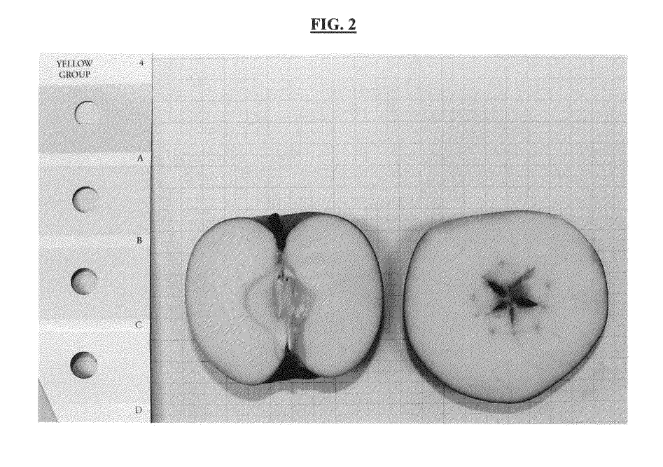

FIG. 3—shows a fruit-bearing tree of ‘KIZURI’, at 12 years of age.

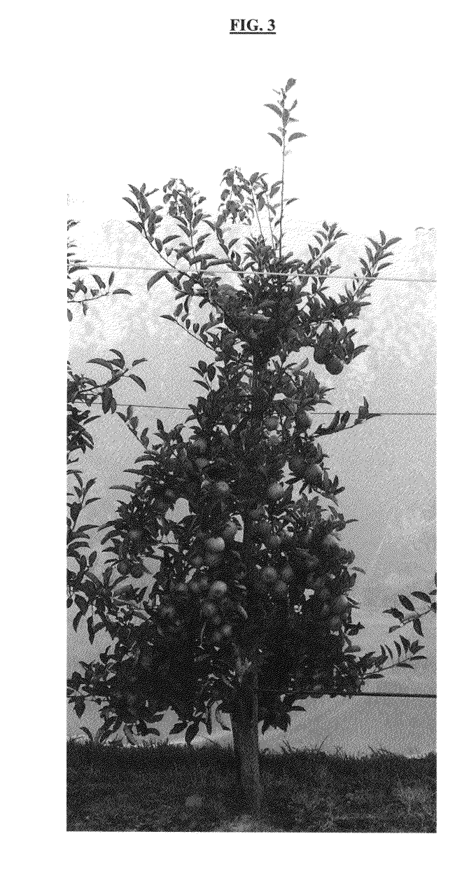

FIG. 4—shows a dormant tree of ‘KIZURI’, at 12 years of age

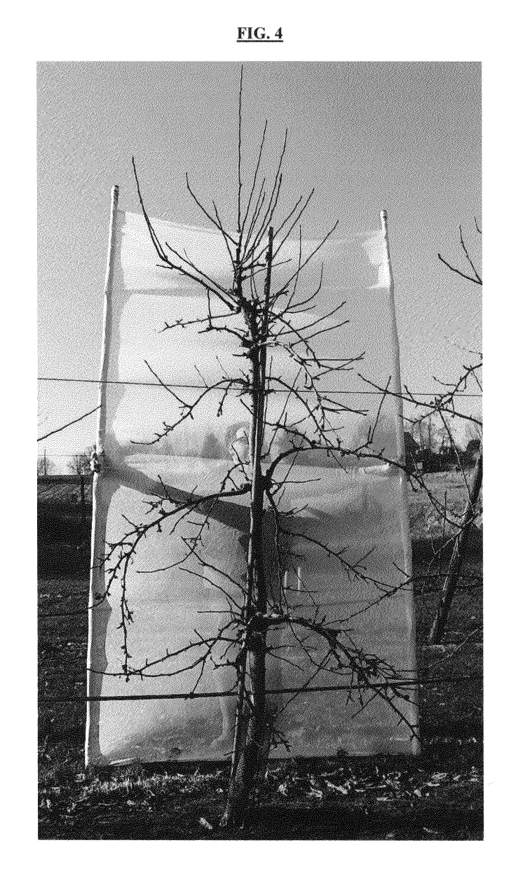

FIG. 5—shows a blooming tree of ‘KIZURI’, at 12 years of age.

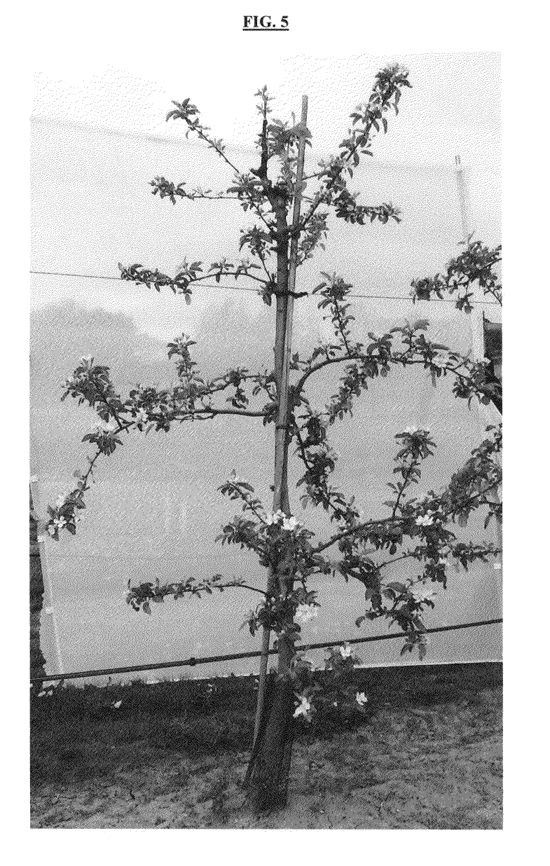

FIG. 6—shows inflorescence of ‘KIZURI’.

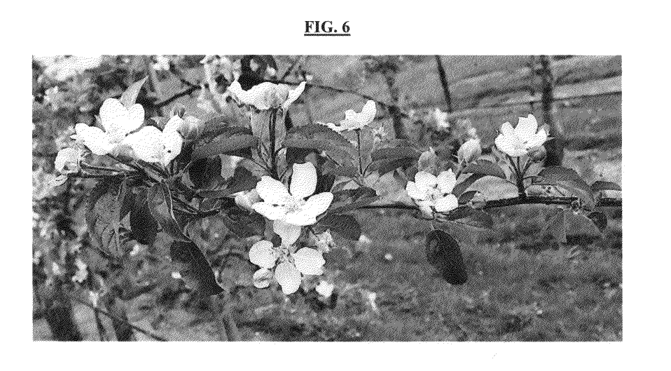

FIG. 7—shows the upper surface (top) and under surface (bottom) view of leaves of ‘KIZURI’.

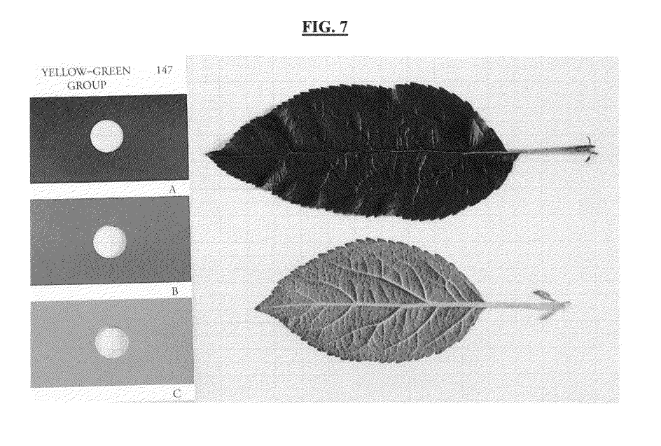

FIG. 8—shows different stages of development of the flowers and the (from left to right) top, side and bottom view of a fully expanded flower of ‘KIZURI’.

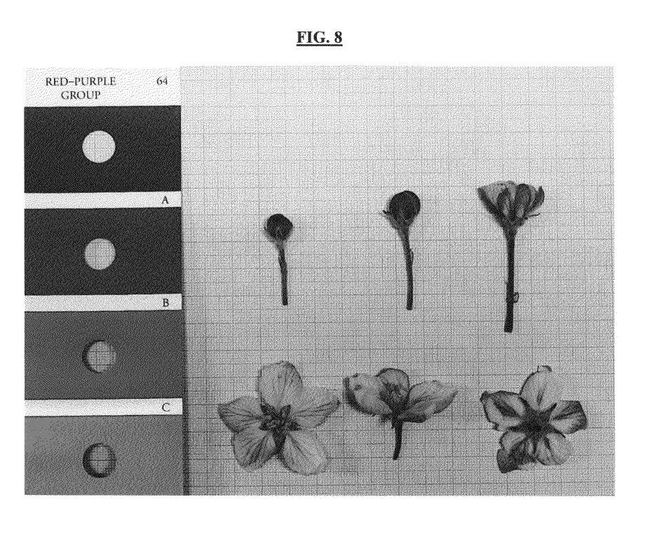

FIG. 9—shows different parts of a flower of ‘KIZURI’ (from left to right: upper and lower surface of petal, upper and lower surface of sepal, stamen, pistil).

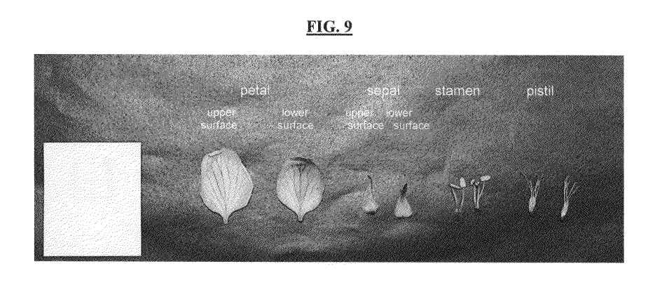

### DETAILED BOTANICAL DESCRIPTION

The new Malus variety ‘KIZURI’ has not been observed under all possible environmental conditions. The phenotype of the new variety may vary with variations in environment such as temperature, light intensity, and day length without any change in the genotype of the Apple tree.

The aforementioned photographs, together with the following observations, measurements and values describe trees of ‘KIZURI’ as grown in Rillaar, Belgium, under conditions which closely approximate those generally used in commercial practice.

Unless otherwise stated, the detailed botanical description includes observations, measurements and values based on 12 years old ‘KIZURI’ trees grown in Rillaar, Belgium from 2004 to 2015. Quantified measurements are expressed as an average of measurements taken from a number of trees of ‘KIZURI’. The measurements of any individual tree, or any group of trees, of the new variety may vary from the stated average.

Color references are made to The Royal Horticultural Society Colour Chart (R.H.S.), (4^{th} edition, 2001), except where general colors of ordinary significance are used. Color values were taken under daylight.

All of the trees of ‘KIZURI’, insofar as they have been observed, have been identical in all the characteristics described below.
Classification:
   Botanical.—Malus domestica.
Parentage:
   Female or seed parent.—Malus variety designated ‘GOLDEN DELICIOUS’ (unpatented).
   Male or pollen parent.—Malus variety designated ‘NY75413-30’ (unpatented).
Propagation: Grafting onto rootstocks
Growing conditions:
   Light intensities.—Full sunlight.
   Temperature.—During day, grown in range of 2° C. to 23° C., and during evening, grow in range of −5 ° C. to 13° C. (based on the average month temperature).
Tree:
   Age.—Observed trees were 12 years old.
   Vigor.—medium to strong.
   Form.—ramified, spreading.
   Habit.—A medium-sized tree with one trunk and 9+/−3 leader(s); main branches spreading; crown symmetrical.
   Branching habit.—Main branches angle is 81+/−25 ° with respect to trunk if allowed to grow naturally.
   Density.—about 2200 trees per hectare.
   Cropping behavior.—normal beginning production; medium to high productivity; medium to abundant flowering; little biennial bearing.
   Type of bearing.—on long shoots.
   Production.—About 16.9 kg/tree in 2013.
   Size at maturity.—Height: About 262+/−11 cm Spread: About 165+/−22 cm.
   Trunk.—Height (up to leaders): About 92+/−12 cm Diameter: About 9+/−2 cm Texture: rough Bark color: Primarily RHS 197C, with underbark RHS 166C Trunk Lenticels: Length: About 8.0+/−2.9 mm Width: About 1.3+/−0.3 mm Color: RHS 161B Density: About 2.8+/−0.9 n° per cm².
   Branches.—Number per tree: About 74+/−25 Length: maximum 94+/−18 cm; minimum 6+/−3 cm. Diameter (at 2 years): About 19+/−4 mm Surface texture: slightly rough Pubescence: none Color: Mature (after about 3 year old): RHS 200B New Growth: RHS 166A Internode length: About 35+/−9 mm Internode diameter: About 9+/−2 mm Branch lenticels: Length: About 2.9+/−0.8 mm Width: About 1.1+/−0.2 mm Color: RHS 161C Density: About 4.3+/−1.7 n° per cm².
   Spur.—Present: Yes Distance between each spur: On the 2 and 3 year old branches, the distance is about 3+/−1 cm Diameter of each spur: About 5+/−1 mm Number of fruit per spur: About 1.4+/−1.1.
Foliage:
   Arrangement.—Alternate, simple, petiolated.
   Lamina.—Size: Length: About 103+/−15 mm (fully expanded leaf) Width: About 55+/−7 mm (fully expanded leaf) Length/width ratio: 1.9+/−0.2 Overall Shape: obtuse, petiolated Base shape: obtuse Apex shape: acute Margin: biserrate Texture: Upper surface: smooth, glabrous Under surface: smooth, medium pubescence (white) Attitude in relation to shoot: upwards Color (mature leaves): Upper surface: RHS 147A Under surface: RHS 138B Color (immature leaves): Upper surface: RHS 143B Under surface: RHS 143C.
   Venation.—Type: pinnate Color: RHS 144D.
   Petiole.—Length: About 33+/−7 mm Diameter: About 1.8+/−0.2 mm Texture: smooth, medium pubescence (white) Color: RHS 144B (upper surface), RHS 144C (lower surface), RHS 59B (at the petiole base, upper and lower surface).
   Stipule.—Arrangement: free standing Length (distance of stipules from basal attachment of petiole): About 6.5+/−1.7 mm Width: About 1.5+/−0.5 mm.
Inflorescence:
   Blooming time.—early to medium (after Idared and Boskoop, before Jonagored).
   Blooming period.—About 1 week.
   Fragrance.—slight.
   Type.—corymb.
   Number of flowers per inflorescence.—About 4.8+/−0.9.
   Inflorescence size.—Diameter: About 86+/−9 mm Depth: About 57+/−6 mm.
   Buds.—Terminal Buds: Number per spur: About 1 Shape: a cone with an ellipsoidal base Length: About 10.3+/−2.3 mm Width: About 5.2+/−0.8 mm Texture: smooth, high pubescence Color: Apex RHS N186B, and base RHS 187A Scales: Number: About 7.1+/−2.0 Overall shape: triangular, folded double around bud Apex shape: 3 blunt points Base shape: straight, fully grown together at base Color: Upper: RHS 187A Lower: RHS 151B Lateral Buds: Number per spur: About 3.9+/−1.9 Shape: a cone with an ellipsoidal base Length: About 5.8+/−1.6 mm Width: About 3.5+/−0.5 mm Texture: smooth, slight pubescence at apex Color: Apex RHS 187B, and base RHS N186D Scales: Number: About 5.4+/−0.9 Overall shape: triangular, folded double around bud Apex shape: acute Base shape: straight, fully grown together at base Color: Upper: RHS N186C Lower: RHS 151D.
   Petals.—Arrangement: intermediate Number per flower: 5 Size: Length: About 21+/−3 mm Width: About 16+/−2 mm Length/width ratio: 1.3+/−0.1 Overall shape: ovate Apex shape: rounded Base shape: rounded Texture (upper surface): smooth, no hairs Texture (lower surface): smooth, no hairs Margin: entire Color (upper surface): Apex RHS 69D, RHS 64D veined; base RHS 69D Color (lower surface): Apex RHS 69D, RHS 64C veined; base RHS 69D.
   Sepals.—Number per flower: 5 Size: Length: About 9.0+/−1.1 mm Width: About 4.5+/−0.5 mm Length/width ratio: 2.0+/−0.3 Overall shape: triangular Apex shape: aristate Base shape: truncate Texture (upper surface): smooth, hairy Texture (lower surface): smooth, hairy Margin: entire Color (upper surface): Apex RHS 178B, base RHS 144B Color (lower surface): Apex RHS 178B, base RHS 143B.
   Pedicel.—Length: 27+/−5 mm Diameter: 1.6+/−0.2 mm Texture: smooth, hairy (white hairs) Color: RHS 144B; a line of RHS 178B.
Fruit:
   Keeping quality.—It can be stored in cold temperature conditions for up to 6 months (normal atmosphere). It has a shelf life of about 5 weeks.
   Maturity when described.—Ripe for eating.
   Maturity period after full bloom.—About 5.5 months after full bloom.
   Date of first and last picking (harvest).—first 2 weeks of October.
   Type.—Pome.
   General shape.—oblong.
   Average weight.—About 266+/−44 g.
   Fruit size.—Average height: About 74+/−5 mm Average diameter (at widest point): About 84+/−4 mm Position of maximum diameter: middle Height/thickness ratio: 0.9+/−0.1.
   Stem.—Length: About 23+/−5 mm Diameter: About 2.5+/−0.4 mm Color: RHS 199A, RHS 151A (both full length).
   Stalk cavity.—Depth: About 15.5+/−3.2 mm Width: About 34.3+/−3.7 mm.
   Eye basin.—Depth: About 12.3+/−2.7 mm Width: About 32.8+/−4.7 mm Crowning at calyx end: weak to moderate Position of sepals: Half-closed Calyx tube: closed to partly open.
   Skin.—Thickness: 0.2+/−0.1 mm Texture: tough Bloom: absent Greasiness: absent Firmness (at picking time): 7.6+/−0.9 kg/cm² Overcolor color: RHS 45A Percentage of skin surface with overcolor color: About 50 to 75% Pattern of overcolor: only solid flush Intensity of overcolor: medium to low Ground color: RHS 1C Skin Lenticels: Length: About 1.4+/−0.2 mm Width: About 1.1+/−0.2 mm Color: RHS 197D Density: About 3.0+/−2.0 n° per cm².
   Flesh.—Color: RHS 4D Texture: firm, crisp and juicy Aroma: strong Eating quality: excellent Sugar content (at picking time): 14.5+/−1.4 Brix Acidity/Starch (at picking time) 5.6+/−0.3 g/1 malic acid Core: Symmetry of core: round Distinctness of core lines: weak Locules: Number (per fruit): 5 Length: About 10+/−1 mm Width: About 4+/−1 mm Form: teardrop shape.
Seeds:
   Number per fruit.—About 5.4+/−3.8.
   Number per locule.—About 1.2+/−0.8.
   Shape.—teardrop shape.
   Length.—About 9.6+/−1.1 mm.
   Width.—About 5.0+/−0.4 mm.
   Texture.—smooth.
   Color.—RHS 166A and RHS 166B.
Reproductive organs:
   Androecium.—Stamen: Number per flower: 19.2+/−1.1 Length: About 8.5+/−1.9 mm Anther: Length: About 3.1+/−0.4 mm Color: RHS IOC Filaments: Length: About 7.3+/−1.5 mm Color: RHS N155C Pollen: Amount: high Color: RHS 6B Pollination Requirements: cross pollination.
   Gynoecium.—Pistils: Quantity: 5 Length: About 14.0+/−1.2 mm Color: RHS 144C Stigmas: Color: RHS 144C Ovary: Length: About 4.7+/−0.7 mm. Width: About 1.7+/−0.5 mm Color: RI-IS 144C.
Use: Fresh market.
Disease/pest resistance: no known resistance to pests and diseases.
Disease/pest susceptibility: tolerant to canker (Nectria galligena), powdery mildew (Podosphaera leucotricha) and fire blight (Erwinia amylovora); susceptible to scab (Venturia inaequalis) and wooly apple aphid (Eriosoma lanigerum).
Winter hardiness: Tolerant to temperatures down to −10° C. without observed damage to wood and buds of dormant Apple trees.
Drought/heat tolerance: Tolerant to temperatures up to 40° C., growth is limited by drought periods without irrigation.
Shipping/storage characteristics: not sensitive to bruising; good storability under ULO-conditions (maintains high quality for up to 6 months).

1. A new and distinct variety of Malus domestica apple tree named ‘KIZURI’, as illustrated and described herein.
